(19) United States
(12) Patent Application Publication
(10) Pub. No.: US 2019/0381515 A1
(43) Pub. Date: Dec. 19, 2019

(54) APPARATUS FOR MANIPULATING MAGNETIC PARTICLES

(71) Applicant: SHIMADZU CORPORATION, Kyoto-shi (JP)

(72) Inventors: Akira MURAMATSU, Kyoto-shi (JP); Masamitsu SHIKATA, Kyoto-shi (JP); Ayaka YAMANO, Kyoto-shi (JP)

(73) Assignee: SHIMADZU CORPORATION, Kyoto-shi (JP)

(21) Appl. No.: 16/401,646

(22) Filed: May 2, 2019

(30) Foreign Application Priority Data
Jun. 14, 2018 (JP) 2018-113372

(51) Int. Cl. B03C 1/28; B03C 1/01; G01N 33/543

(52) U.S. Cl. CPC B03C 1/288; B03C 1/01; B03C 2201/26; B03C 2201/18; B03C 2201/20; G01N 33/54333

(57) ABSTRACT

A magnet is disposed close to and outside a container held by a container holding part, and relatively moves along the container, so as to move the magnetic particles in the container by a magnetic force. A housing accommodates the magnet and the container holding part in the housing in a state where a position of the magnet relatively moving along the container is not visually recognizable from the outside. A display unit is provided on the housing so as to be visually recognizable from the outside and has light emitting regions that can emit light. A processing step execution unit sequentially executes a plurality of processing steps by relatively moving the magnet. When the plurality of processing steps are sequentially executed, a light emission control unit causes the light emitting regions to emit light according to a position of the magnet.

### CROSS REFERENCE TO RELATED APPLICATION

This application claims priority to Japanese Patent Application No.2018-113372 filed on Jun. 14, 2018, the entire disclosure of which is incorporated by reference herein.

### BACKGROUND OF THE INVENTION

### Field of the Invention

The present invention relates to an apparatus for manipulating magnetic particles for moving magnetic particles in a state where a target substance is fixed to the magnetic particles in a tubular device in which a gel-like medium layer and a liquid layer are alternately stacked in a container and the magnetic particles are loaded.

### Description of the Related Art

In medical examinations, food safety and hygiene management, monitoring for environmental conservation, and the like, it is required to extract a target substance from a sample containing various kinds of contaminants and use it for detection and reaction. For example, in a medical examination, it is necessary to detect, identify, and quantify nucleic acids, proteins, sugars, lipids, bacteria, viruses, radioactive substances, and the like contained in blood, serum, cells, urine, feces, and the like separated and obtained from animals and plants. In these examinations, it is sometimes necessary to separate and purify a target substance in order to eliminate adverse influences, such as background caused by contaminants.

In order to separate and purify a target substance in a sample, a method using magnetic particles having a chemical affinity with the target substance and a molecular recognition function on a surface of a magnetic substance having a particle size of about 0.5 μm to about ten and several pm has been developed and put to practical use. In this method, processes of separating and recovering magnetic particles from a liquid phase by magnetic field operation after a target substance is fixed to a surface of the magnetic particles, and, if necessary, dispersing the recovered magnetic particles into a liquid phase of a cleaning solution and the like, and separating and recovering the magnetic particles from the liquid phase are repeatedly performed. After the above, the magnetic particles are dispersed in an eluate, so that the target substance fixed to the magnetic particles is liberated in the eluate, and the target substance in the eluate is recovered. By using magnetic particles, it is possible to recover a target substance by a magnet, and therefore it has features advantageous for automation of chemical extraction and purification.

Magnetic particles to which a target substance can be selectively fixed are commercially available as part of a separation and purification kit. In the kit, a plurality of reagents are placed in separate containers, and in use, the user fractionates and dispenses the reagents with a pipette or the like. Devices for automating these pipetting operations and magnetic field operations are also commercially available (International Publication Pamphlet of WO97/44671). On the other hand, there has been proposed a method in which, instead of a pipetting operation, a tubular device in which a liquid layer, such as a dissolving and fixing solution, a cleaning solution, an eluate, and the like and a gel-like medium layer are alternately stacked in a tubular container, such as a capillary, is used, and magnetic particles are moved in a longitudinal direction of the container in the tubular device, so that a target substance is separated and purified (International Publication Pamphlet of WO2012/086243).

In the configuration in which magnetic particles are moved in a tubular container as described above, a magnet as a magnetic field applying part provided outside the container is moved along a longitudinal direction of the container, so that a magnetic field is changed. Following the change in the magnetic field, the magnetic particles also move along the longitudinal direction of the container, and the magnetic particles sequentially move through alternately-stacked liquid layer and gel-like medium layer. In the process in which the magnetic particles move through a plurality of liquid layers in this manner, different processing steps are performed in each liquid layer, so that a plurality of processing steps corresponding to positions of the magnet are executed.

When a magnet is moved along a longitudinal direction of a container, if a distance between magnetic particles in the container and the magnet is too far, the magnetic particles cannot be satisfactorily moved along the longitudinal direction of the container. Since the container is generally an elongated shape and warped, in order to keep a distance between magnetic particles in the container and a magnet at a fixed short distance, a configuration of correcting the warp of the container is employed at present. Specifically, a flat abutting surface is pressed against the container from the opposite side to the magnet, so that the container is straightened so as to extend in a straight line.

However, in this case, since the container is not visually recognizable from the outside due to an abutting surface pressed against the container, a position of the magnet moving along the container is not visually recognizable from the outside, either. Since the position of the magnet along the longitudinal direction of the container corresponds to a position of the layer (liquid layer or gel-like medium layer) in which the magnetic particles are present in the container, in a state where the position of the magnet is not visually recognizable from the outside, there is a problem that it is not possible to determine a type of a processing step being executed.

The present invention has been made in view of the above circumstances, and a purpose of the present invention is to provide an apparatus for manipulating magnetic particles that makes it possible to determine a type of a processing step being executed even in a state where the position of the magnet is not visually recognizable from the outside.

### SUMMARY OF THE INVENTION

(1) An apparatus for manipulating magnetic particles according to the present invention is for moving magnetic particles in a state where a target substance is fixed to the magnetic particles in a tubular device in which a gel-like medium layer and a liquid layer are alternately stacked in a container and the magnetic particles are loaded. The apparatus for manipulating magnetic particles includes a container holding part, a magnet, a drive unit, a display unit, a storage unit, a processing step execution unit, and a light emission control unit. The container holding part holds the container. The magnet is disposed close to and outside the container held by the container holding part, and relatively moves along the container, so as to move the magnetic particles in the container by a magnetic force. The drive unit relatively moves the magnet along the container. The housing accommodates the magnet and the container holding part in a state in which the position of the magnet relatively moves along the container is not visually recognizable from the outside. The display unit is provided on the housing so as to be visually recognizable from the outside, and has a light emitting region capable of emitting light. The storage unit holds details of a process step of relatively moving the magnet. The processing step execution unit sequentially executes a plurality of processing steps by relatively moving the magnet. The light emission control unit causes the light emitting region to emit light according to the position of the magnet when the plurality of processing steps are sequentially executed.

According to such a configuration, when a plurality of processing steps are sequentially executed, the light emitting region that emits light according to the position of the magnet is visually recognizable from the outside. Therefore, even when the position of the magnet is not visually recognizable from the outside, it is possible to determine a type of processing being executed.

(2) The liquid layer may include a layer made of a cleaning solution for removing impurities other than the target substance.

According to such a configuration, it is possible to determine whether or not a processing step (cleaning step) for removing impurities by a cleaning solution is being executed even if the position of the magnet is not visually recognizable from the outside. Therefore, it is also possible to accurately determine a timing at which the cleaning processing is performed and a timing at which the cleaning process is not performed, and to take out the container in the middle of any of the processing steps.

(3) The liquid layer may contain a layer made of a reagent acting on the target substance.

According to the above configuration, even if the position of the magnet is not visually recognizable from the outside, whether or not a processing step (reagent step) for causing the reagent to act on the target substance is being executed can be determined. Therefore, it is also possible to accurately determine a timing at which the reagent step is being performed and a timing at which the reagent step is not performed, and to take out the container in the middle of any of the processing steps.

The reagent step may include a restriction enzyme step in which the restriction enzyme for fragmenting a nucleic acid acts on the target substance as a reagent and a reaction enzyme step in which the reaction enzyme for reacting a fragmented nucleic acid acts on the target substance as a reagent. In this case, a timing at which the restriction enzyme step is performed and a timing at which the reaction enzyme step is performed can be accurately determined. Therefore, it is also possible to take out the container, for example, after the restriction enzyme step is performed and before the reaction enzyme step is performed.

(4) The display unit may have a plurality of the light emitting regions that can emit light individually.

According to such a configuration, when a plurality of processing steps are sequentially executed, the plurality of light emitting regions can be caused to emit light individually according to the position of the magnet, so that a type of a processing step being executed can be easily determined.

(5) The display unit may include the plurality of light emitting regions arranged side by side.

According to such a configuration, by causing the plurality of light emitting regions arranged side by side to emit light individually, a process in which a plurality of processing steps are sequentially executed can be displayed in an easy-to-understand manner.

(6) When the plurality of processing steps are sequentially executed, the light emission control unit may cause the light emitting regions corresponding to the respective processing steps to sequentially emit light.

According to such a configuration, by causing the light emitting regions corresponding to the respective processing steps to emit light sequentially, a process in which the plurality of processing steps are sequentially executed can be displayed in an easy-to-understand manner.

(7) The apparatus for manipulating magnetic particles may further include an operation unit that is operated to execute a next processing step while part of processing steps is omitted among a plurality of processing steps sequentially executed by the processing step execution unit. In this case, the light emission control unit may cause the light emitting region corresponding to the processing step to emit light when the next processing step is executed by operation of the operation unit.

According to such a configuration, by operating the operation unit, it is possible to execute a next processing step while part of the plurality of processing steps sequentially executed is omitted. When the next processing step is executed, a light emitting region corresponding to the processing step emits light. Accordingly, even when part of processing steps are omitted, a type of a processing step being executed can be accurately determined.

For example, in the case where the reagent step includes the restriction enzyme step and the reaction enzyme step as described above, it may be desired to cause a nucleic acid to react with the reaction enzyme without fragmenting the nucleic acid by executing the reaction enzyme step while the restriction enzyme step is omitted. In such a case, by operating the operation unit, the reaction enzyme step can be executed while the restriction enzyme step is omitted. Even in such a case, by visually recognizing the light emission in the light emitting region, it can be accurately determined that the reaction enzyme step is being executed while the restriction enzyme step is omitted.

(8) When a specific processing step determined in advance as a processing step that should not be stopped halfway among a plurality of processing steps sequentially executed by the processing step execution unit is executed, the light emission control unit may cause the light emitting region corresponding to the specific processing step to emit light in a mode different from that of the light emitting regions corresponding to other processing steps.

According to the above configuration, it is possible to easily determine whether a processing step being executed is a processing step which should not be stopped halfway or another processing step based on a light emission mode of the light emitting regions. Therefore, during execution of the processing step which should not be stopped halfway, the container can be prevented from being taken out after the processing step is erroneously stopped.

According to the present invention, even in a state where a position of a magnet is not visually recognizable from the outside, it is possible to determine a type of a processing step being executed by visually recognizing a light emitting region that emits light according to the position of the magnet when a plurality of processing steps are sequentially executed.

### BRIEF DESCRIPTION OF THE DRAWINGS

FIG. 1 is a front view showing a configuration example of a tubular device;

FIG. 2 is a cross-sectional view taken along the line A-A of the tubular device of FIG. 1;

FIG. 3 is a front view showing a configuration example of an apparatus for manipulating magnetic particles according to one embodiment of the present invention;

FIG. 4 is a cross-sectional view taken along the line B-B of the apparatus for manipulating magnetic particles of FIG. 3;

FIG. 5A-5C are schematic diagrams for explaining a mode when magnetic particles are manipulated;

FIG. 6 is a schematic front view for explaining the appearance of the apparatus for manipulating magnetic particles; and

FIG. 7 is a block diagram showing an example of an electrical configuration of the apparatus for manipulating magnetic particles.

### DETAILED DESCRIPTION OF THE PREFERRED EMBODIMENTS

1. Tubular Device

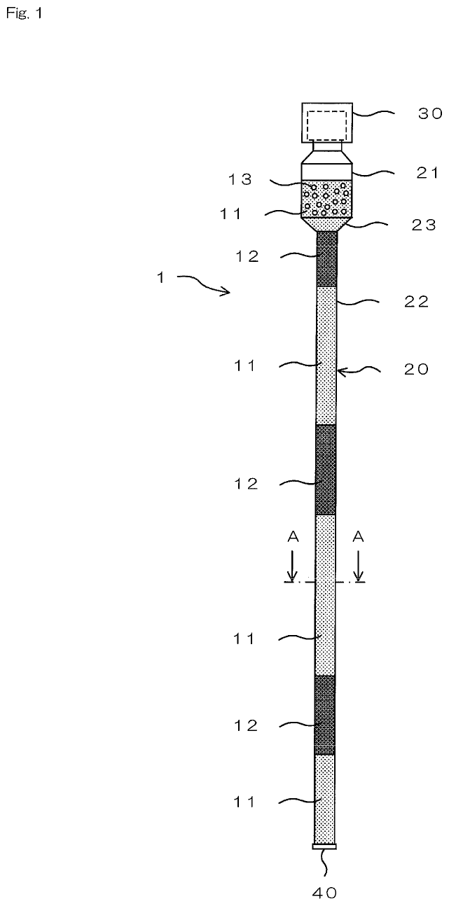

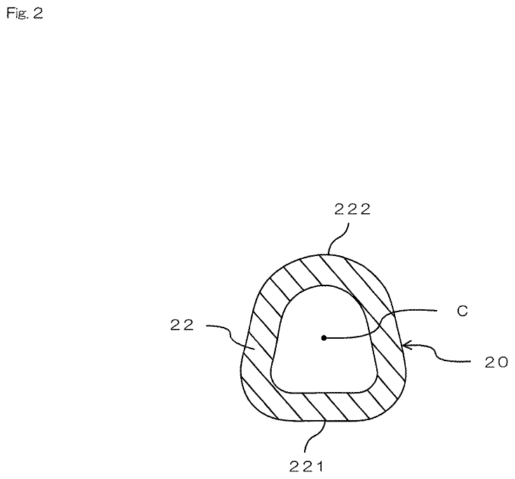

FIG. 1 is a front view showing a configuration example of a tubular device 1. FIG. 2 is a cross-sectional view taken along the line A-A of the tubular device 1 of FIG. 1. The tubular device 1 is for extracting and purifying a target substance from a liquid sample and includes a tubular container 20 extending in a straight line.

In the container 20, a plurality of liquid layers 11 and a plurality of gel-like medium layers 12 are formed. Specifically, the liquid layer 11 is formed in the lowermost part of the container 20, and the gel-like medium layer 12 and the liquid layer 11 are alternately stacked in a longitudinal direction in the upward direction. In this example, four of the liquid layers 11 and three of the gel-like medium layers 12 are alternately formed in the longitudinal direction. However, the present invention is not limited to this configuration, and the numbers of liquid layers 11 and gel-like medium layers 12 can be set optionally.

The uppermost liquid layer 11 of the container 20 is a solution loaded with a large number of magnetic particles 13. The solution is, for example, water, a surfactant, or the like, and a liquid sample containing a target substance is mixed in the solution. The solution may be mixed with the liquid sample and then injected into the container 20. The lowermost liquid layer 11 of the container 20 is an eluate for eluting the target substance in the liquid sample. One or a plurality (in this example, two) of the liquid layers 11 in the middle part of the container 20 is a cleaning solution for removing impurities (contaminants) other than the target substance contained in the liquid sample. These liquid layers 11 are separated from each other by the gel-like medium layer 12. A manipulation (particle manipulation) of moving the magnetic particles 13 from the uppermost part to the lowermost part of the container 20 is performed by changing the magnetic field in a state where the target substance contained in the liquid sample is fixed to the magnetic particles 13. During the manipulation, the target substance is washed with the cleaning solution and then eluted into the lowest eluate.

The magnetic particles 13 are particles on which or in which a target substance, such as a nucleic acid and an antigen, can be specifically fixed. By dispersing the magnetic particles 13 in the uppermost liquid layer 11 of the container 20, the target substance contained in the liquid layer 11 is selectively fixed to the magnetic particles 13.

A method of fixing the target substance to the magnetic particles 13 is not particularly limited, and various known fixation mechanisms, such as physical adsorption and chemical adsorption, can be applied. For example, the target substance is fixed to the surface or inside of the magnetic particles 13 by various intermolecular forces, such as van der Waals force, hydrogen bonding, hydrophobic interaction, ionic interaction, π-π stacking, and the like.

The particle size of the magnetic particles 13 is preferably 1 mm or less, more preferably from 0.1 μm to 500 μm, and still more preferably from 3 to 5 μm. The shape of the magnetic particles 13 is preferably spherical with uniform particle diameters, but may have an irregular shape and a certain particle size distribution so long as particle manipulation is possible. The constituent components of the magnetic particles 13 may be a single substance or a plurality of components.

The magnetic particles 13 may be composed of only a magnetic substance, but preferably those to which a coating for specifically fixing the target substance to the surface of the magnetic substance is applied. Examples of the magnetic material include iron, cobalt, nickel, and compounds, oxides and alloys of these. Specifically, it is possible to use magnetite (Fe₃O₄), hematite (Fe₂O₃ or αFe₂O₃), maghemite (γFe₂O₃), titanomagnetite (xFe₂TiO₄.(1-x) Fe₃O₄), ilmeno hematite (xFeTiO₃.(1-x) Fe₃O₃), pyrrhotite (Fe₁₋ₓS(x=0 to 0.13) . . . Fe₂S₈(x to 0.13)), greigite (Fe₃S₄), geothite (αFeOOH), chromium oxide (CrO₂), permalloy, alconi magnet, stainless steel, samarium magnet, neodymium magnet, and barium magnet.

Examples of the target substance selectively fixed to the magnetic particles 13 include organism-derived substances, such as nucleic acids, proteins, sugars, lipids, antibodies, receptors, antigens, ligands and the like, and the cells themselves. When the target substance is an organism-derived substance, the target substance may be fixed to the inside of the magnetic particles 13 or a particle surface by molecular recognition or the like. For example, when the target substance is a nucleic acid, magnetic particles or the like having silica coating on its surface are preferably used as the magnetic particles 13. In a case where the target substance is an antibody (for example, a labeled antibody), a receptor, an antigen, a ligand or the like, the target substance can be selectively fixed to the particle surface by an amino group, a carboxyl group, an epoxy group, avidin, biotin, digoxigenin, protein A, protein G, or the like on the surface of the magnetic particles 13. As the magnetic particles 13 to which a specific target substance can be selectively fixed, commercially available products of magnetic beads enclosed in, for example, Dynabeads (registered trademark) sold by Thermo Fisher Scientific or the like can also be used.

In a case where the target substance is a nucleic acid, the cleaning solution is preferably one that may liberate components (for example, proteins, carbohydrates, and the like) other than the nucleic acid contained in the liquid sample, or impurities, such as reagents used in treatments like nucleic acid extraction and the like, into the cleaning solution while maintaining a state in which the nucleic acid is fixed to the surface of the magnetic particles 13. As the cleaning solution, for example, a high salt aqueous solution of sodium chloride, potassium chloride, ammonium sulfate, or the like, or an alcoholic aqueous solution of ethanol, isopropanol, or the like can be used.

As an eluate (nucleic acid eluate) for eluting a nucleic acid, a buffer solution containing water or a low concentration salt can be used. Specifically, a Tris buffer solution, a phosphate buffer solution, distilled water, and the like can be used, and it is general to use a 5 to 20 mM Tris buffer solution adjusted to pH 7 to 9. By dispersing the magnetic particles 13 having a fixed nucleic acid in the eluate, it is possible to liberate and elute the nucleic acid in the nucleic acid eluate. The recovered nucleic acid can be subjected to analysis, reaction, and the like after manipulations, such as concentration and drying, are performed as necessary.

The gel-like medium layer 12 is in a gel or paste form before particle manipulation. The gel-like medium layer 12 is preferably insoluble or poorly soluble in the adjacent liquid layer 11 and made of a chemically inactive substance. Here, insoluble or poorly soluble in liquid means that solubility in liquid at 25° C. is about 100 ppm or less. The chemically inactive substance refers to a substance that does not exert a chemical influence on the liquid layer 11, the magnetic particles 13, or a substance fixed to the magnetic particle 13 in contact with the liquid layer 11 or manipulation of the magnetic particle 13 (that is, manipulation of moving the magnetic particles 13 in the gel-like medium layer 12).

A material, composition, and the like of the gel-like medium layer 12 are not particularly limited, and may be a physical gel or a chemical gel. For example, as described in WO2012/086243, a water-insoluble or poorly water-soluble liquid substance is heated, a gelling agent is added to the heated liquid substance to completely dissolve the gelling agent. After that, the liquid substance is cooled to a sol-gel transition temperature or less, so that a physical gel is formed.

The liquid layer 11 and the gel-like medium layer 12 can be loaded into the container 20 by an appropriate method. In a case of using the tubular container 20 as in the present embodiment, it is preferable that an opening at one end (for example, a lower end) of the container 20 is sealed prior to loading, and the liquid layer 11 and the gel-like medium layer 12 are sequentially loaded from an opening portion on the other end (for example, an upper end).

The capacity of the liquid layer 11 and the gel-like medium layer 12 loaded in the container 20 can be appropriately set according to an amount of magnetic particles 13 to be manipulation, a type of manipulation, and the like. In the case where the plurality of liquid layers 11 and the gel-like medium layers 12 are provided in the container 20 as in the present embodiment, the capacity of each layer may be the same or different. The thickness of each layer can also be appropriately set. In consideration of operability and the like, the thickness of each layer is preferably about 2 to 20 mm, for example.

The uppermost portion of the container 20 is a bulging portion 21 having an inner diameter and an outer diameter larger than the other portions. An upper surface of the bulging portion 21 is an opening portion, and the opening portion can be sealed with a cap 30 detachable from the bulging portion 21. With the cap 30 removed, a liquid sample is injected into the bulging portion 21, so that the uppermost liquid layer 11 of the container 20 is formed.

A portion of the container 20 below the bulging portion 21 is a linear portion 22 whose cross-sectional shape orthogonal to the longitudinal direction is a constant shape as shown in FIG. 2. The bulging portion 21 and the linear portion 22 are connected by a tapered portion 23 tapering from the bulging portion 21 side toward the linear portion 22 side. An opening is formed at a lower end of the linear portion 22 (the bottom surface of the container 20), and the opening is sealed by a film member 40. The target substance eluted into the eluate which is the lowermost liquid layer 11 of the container 20 can be sucked out into a pipette by inserting the pipette into the eluate so as to penetrate the film member 40. The film member 40 is formed of, for example and not limited to, aluminum.

The material of the container 20 is not particularly limited as long as it allows the magnetic particles 13 to move in the container 20 and can hold the liquid layer 11 and the gel-like medium layer 12. In order to move the magnetic particles 13 in the container 20 by performing an operation (magnetic field operation) of changing the magnetic field from outside the container 20, a magnetically permeable material, such as plastic, is preferably used, and, for example, polyolefin, such as polypropylene and polyethylene, fluorine-based resin, such as tetrafluoroethylene, and resin materials, such as polyvinyl chloride, polystyrene, polycarbonate, and cyclic polyolefin, can be used. As a material of the container 20, in addition to the above-mentioned materials, ceramic, glass, silicone, nonmagnetic metal, and the like can also be used. In order to enhance the water repellency of an inner wall surface of the container 20, coating with fluorine-based resin, silicone, or the like may be performed.

As the shape of the container 20, as shown in FIG. 2, a cross-sectional shape (a cross-sectional shape orthogonal to the longitudinal direction) of the linear portion 22 below the bulging portion 21 of the container 20 is a shape asymmetrical with respect to the center C. Specifically, the outer peripheral surface on the front side of the linear portion 22 is a flat surface 221, and the outer peripheral surface on the rear side on the opposite side with respect to the center C is a convex curved surface 222. However, the shape of the container 20 is not limited to the above-described shape, and for example, the cross-sectional shape of the linear portion 22 may be a shape symmetrical with respect to the center C (for example, a circle or the like).

2. Apparatus for Manipulating Magnetic Particles

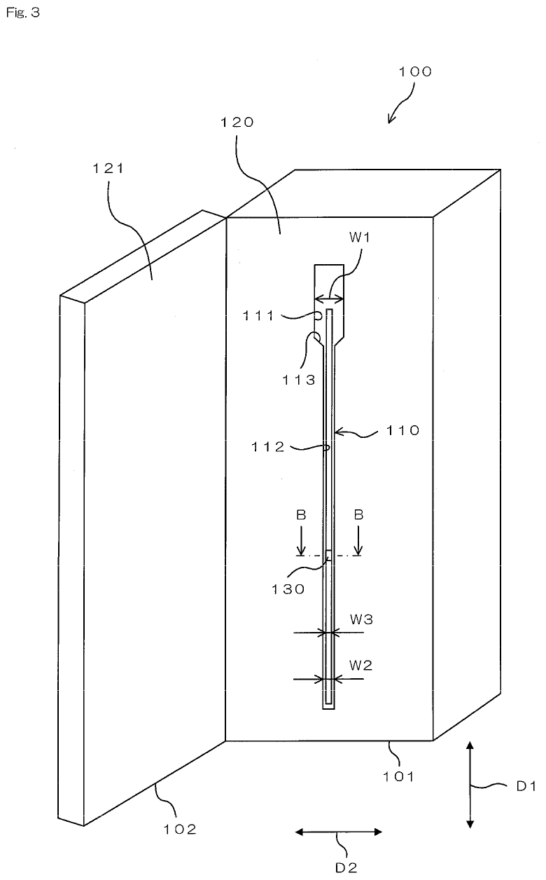

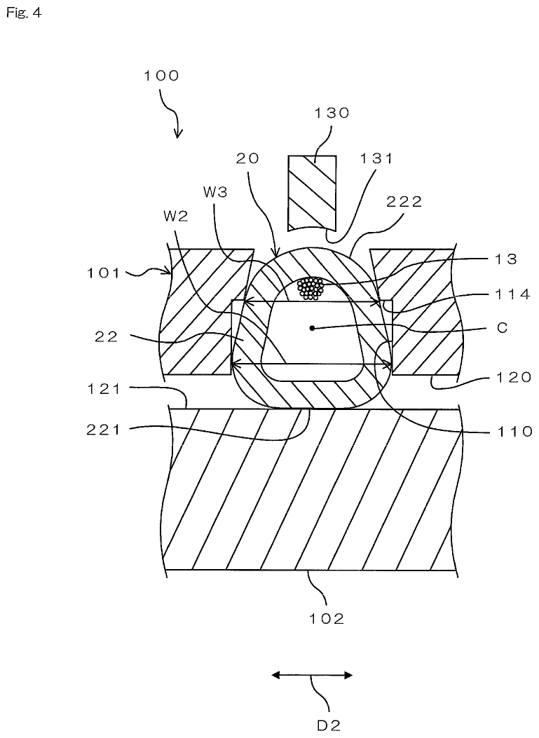

FIG. 3 is a front view showing a configuration example of an apparatus 100 for manipulating magnetic particles according to one embodiment of the present invention. FIG. 4 is a cross-sectional view taken along the line B-B of the apparatus 100 for manipulating magnetic particles of FIG. 3; This apparatus 100 for manipulating magnetic particles (hereinafter referred to as the “apparatus 100”) is used in a state in which the tubular device 1 shown in FIGS. 1 and 2 is fixed, and is for performing particle manipulation to a target substance included in a liquid sample in the container 20 of the tubular device 1.

An outer shape of the apparatus 100 is partitioned by a housing 100a. The housing 100a includes a main body 101 in which a container holding part 110 for holding the tubular device 1 is formed and a container pressing part 102 for pressing and fixing the container 20 of the tubular device 1 held by the container holding part 110. In this example, the container pressing part 102 is configured with a door rotatably attached to the main body 101 by a hinge (not shown). However, as long as the tubular device 1 held by the container holding part 110 can be fixed, the container pressing part 102 is not limited to a configuration that can be rotated with respect to the main body 101, and may have a configuration slidable with respect to the main body 101, a configuration detachable with respect to the main body 101, or the like.

The container holding part 110 is configured with a concave portion formed on a front surface 120 of the main body 101. The container holding part 110 is formed so that a first accommodating portion 111 accommodating the bulging portion 21 of the container 20 of the tubular device 1 and the second accommodating portion 112 accommodating the linear portion 22 continuously extend in a vertical direction D1. In addition, the container holding part 110 has a width in a lateral direction D2, which is orthogonal to the direction (the vertical direction D1) in which the linear portion 22 extends and is parallel to the front surface 120 of the main body 101, that corresponds to a width corresponding to the tubular device 1.

Specifically, a width W1 in the lateral direction D2 of the first accommodating portion 111 is slightly larger than a width of the bulging portion 21 of the container 20. On the other hand, a width W2 of the second accommodating portion 112 in the lateral direction D2 is slightly larger than a width of the linear portion 22 of the container 20 and smaller than the width of the bulging portion 21. Further, the first accommodating portion 111 and the second accommodating portion 112 are connected by a throttle portion 113 which is inclined at an angle corresponding to the tapered portion 23 of the container 20. In this manner, in a state in which the container 20 is accommodated in the container holding part 110, the tapered portion 23 of the container 20 is caught by the throttle portion 113 of the container holding part 110 and is held in a suspended state.

As shown in FIG. 4, the container 20 is accommodated in the container holding part 110 such that the flat surface 221 extends in the lateral direction D2 and the convex curved surface 222 is positioned closer to a back surface side relative to the flat surface 221. A step portion 114 is formed on the inner surface of the second accommodating portion 112 of the container holding part 110 so as to project inward from both sides in the lateral direction D2. A width W3 of the step portion 114 in the lateral direction D2 of the first accommodating portion 111 is smaller than the width W2 on the front surface 120 side and smaller than the width of the linear portion 22 of the container 20 in the lateral direction D2.

Therefore, the linear portion 22 of the container 20 accommodated in the container holding part 110 from the front surface 120 side is brought into a state where its convex curved surface 222 side abuts on the step portion 114. At this time, the flat surface 221 of the container 20 is projected more forward from the container holding part 110 than the front surface 120 of the main body 101. When the door constituting the container pressing part 102 is closed in this state, an abutting surface 121 facing the front surface 120 of the main body 101 can be caused to abut on and press the flat surface 221 of the container 20 to the rear surface side as shown in FIG. 4. In this manner, the linear portion 22 of the container 20 is sandwiched between the abutting surface 121 and the step portion 114, and the linear portion 22 can be firmly fixed.

The rear side of the container holding part 110 is open, and a magnet 130 is disposed so as to face the container holding part 110. The magnet 130 is close to the container 20 held by the container holding part 110 from an outer side (rear surface side). The magnet 130 is made of a permanent magnet and is held slidably along the vertical direction D1.

The magnet 130 attracts the magnetic particles 13 in the container 20 with a magnetic force. In this manner, the magnetic particles 13 are collected on the convex curved surface 222 side as shown in FIG. 4. By moving the magnet 130 in the vertical direction D1 with the magnetic particles 13 attracted to the magnet 130 side, the magnetic particles 13 in the container 20 can be moved in the vertical direction D1 by the magnetic force.

As described above, the magnet 130 constitutes a magnetic field applying part that moves the magnetic particles 13 in the container 20 by changing the magnetic field. The magnet 130 can be slid by a drive unit, such as a motor. In the example of FIG. 4, a facing surface 131 of the magnet 130 facing the container 20 is configured with a concave curved surface. The facing surface 131 is a concave curved surface having a radius of curvature corresponding to the convex curved surface 222 of the container 20. However, the facing surface 131 is not limited to the one configured with a concave curved surface, and may be configured with, for example, a flat surface or the like.

The shape, size and material of the magnet 130 are not particularly limited as long as the manipulation of the magnetic particles 13 is possible. As the magnet 130, it is also possible to use an electromagnet other than using a permanent magnet. Further, a plurality of the magnets 130 may be provided. The magnet 130 may be configured to change the magnetic field by moving relative to the container 20, and has a configuration not limited to the one in which the magnet 130 moves as in the present embodiment, and may have a configuration in which the container 20 moves.

3. Manipulation of Magnetic Particles

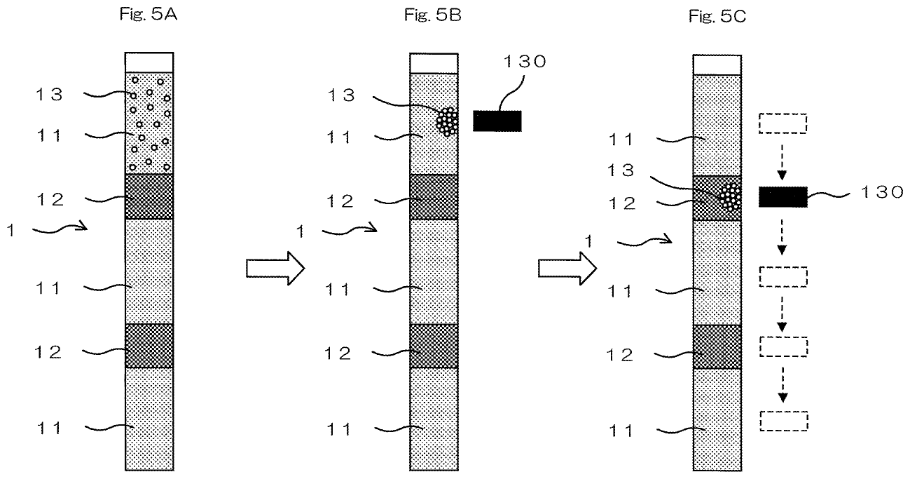

FIG. 5 is a schematic diagram for describing a mode when the magnetic particles 13 are manipulated. In FIG. 5, the shape of the tubular device 1 is simplified for clarity of explanation. In FIG. 5A, the uppermost liquid layer 11 of the container 20 contains a large number of magnetic particles 13. By dispersing the magnetic particles 13 in the liquid layer 11 as described above, the target substance contained in the liquid layer 11 is selectively fixed to the magnetic particles 13.

After the above, as shown in FIG. 5B, when the magnet 130, which is a magnetic force source, is brought close to the outer peripheral surface of the container 20, the magnetic particles 13 to which the objective substance is fixed are gathered on the magnet 130 side (the convex curved surface 222 side) in the container 20 by the action of the magnetic field. Then, as shown in FIG. 5C, when the magnet 130 is moved in the longitudinal direction (vertical direction) of the container 20 along the outer peripheral surface of the container 20, following the change of the magnetic field, the magnetic particles 13 also move along the longitudinal direction of the container 20, and sequentially move in the alternately stacked liquid layer 11 and gel-like medium layer 12.

Most of the liquid physically adhered to the surroundings of the magnetic particles 13 as droplets desorbs from the surface of the magnetic particles 13 when the magnetic particles 13 enter the inside of the gel-like medium layer 12. The gel-like medium layer 12 is pierced as the magnetic particles 13 enter and move into the gel-like medium layer 12. However, due to a self-repairing action by a restoring force of gel, holes of the gel-like medium layer 12 are immediately blocked. Therefore, almost no liquid flows into the gel-like medium layer 12 through a through hole formed by the magnetic particles 13.

By dispersing the magnetic particles 13 in the liquid layer 11 and bringing the magnetic particles 13 into contact with the liquid in the liquid layer 11, fixation of the target substance to the magnetic particles 13, cleaning operation for removing impurities (contaminants) adhered to the surface of the magnetic particles 13, reaction of the target substance fixed to the magnetic particles 13, elution of the target substance fixed to the magnetic particles 13 into liquid, and the like are performed.

4. Appearance of Apparatus for Manipulating Magnetic Particles

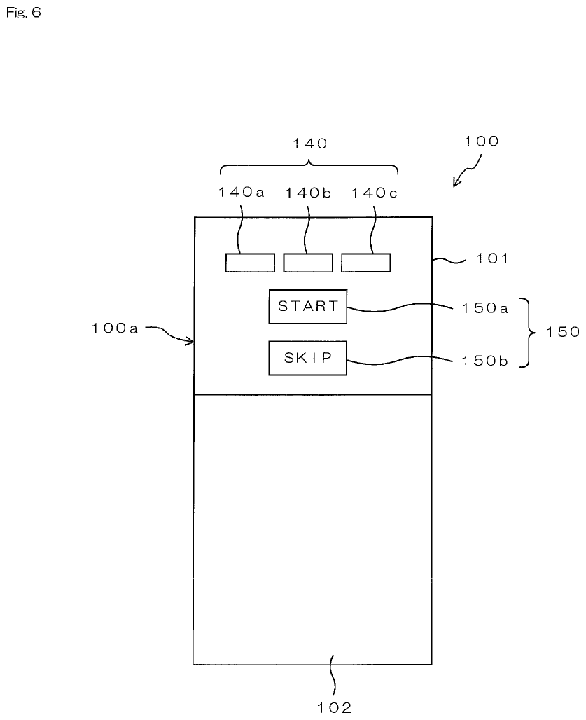

FIG. 6 is a schematic front view for explaining the appearance of the apparatus 100 for manipulating magnetic particles. As shown in FIG. 6, in a state in which the door as the container pressing part 102 is closed and the container 20 of the tubular device 1 is pressed by the container pressing part 102 and fixed in the main body 101, the front of the container 20 is covered by the container pressing part 102. In the state shown in FIG. 6, the magnet 130 and the container holding part 110 are accommodated inside the housing 100a (the main body 101 and the container pressing part 102), and the periphery of the container 20 is completely covered by the housing 100a. For this reason, the magnet 130 relatively moving along the container 20 is not visually recognizable from the outside.

When the magnetic particles 13 are sequentially moved to the plurality of liquid layers 11 by moving the magnet 130, different processing steps are performed in each of the liquid layers 11. In the present embodiment, in a state where the magnet 130 faces the liquid layer 11 in the uppermost part of the container 20, a dissolving step is performed by stirring a solution mixed with the target substance to dissolve the target substance. In a state in which the magnet 130 faces the liquid layer 11 at the bottom part of the container 20, an eluting step for eluting the target substance into the eluate is performed. In a state in which the magnets face one or a plurality (two in this example) of the liquid layers 11 in the middle part of the container 20, a cleaning step for removing impurities other than the target substance is performed. As described above, in the present embodiment, a plurality of processing steps (dissolving step, cleaning step, eluting step) corresponding to positions of the magnet 130 are sequentially executed.

A display unit 140 having a plurality of light emitting regions 140a to 140c is provided on the front surface of the housing 100a. Each of the light emitting regions 140a to 140c includes, for example, a light emitting diode (LED), and can emit light individually. Since the display unit 140 is provided on the front surface of the housing 100a, the user can visually recognize the light emitting regions 140a to 140c of the display unit 140 from the outside with respect to the housing 100a. However, as long as the display unit 140 is visually recognizable from the outside of the housing 100a, the display unit 140 may be provided not only on the front surface of the housing 100a but also on other outer surfaces (a side surface, an upper surface, and the like) of the housing 100a. The way of displaying is not limited to one performed by the LEDs arranged side by side as described above, and may be one using a liquid crystal display or the like, or one performed in a progress bar form. At this time, the display of the progress bar may be associated not only with the processing step, and also with processing time. In this case, a length of the progress bar may correspond to the processing time. The plurality of light emitting regions 140a to 140c do not need to be associated with each processing step. Further, the number of light emitting regions may be one, and for example, the light emitting region may emit light in a color corresponding to a processing step being executed.

On the front surface of the housing 100a, an operation unit 150, such as a start key 150a and a skip key 150b, that can be operated by the user is provided in addition to the display unit 140. Like the display unit 140, the operation unit 150 is not limited to one provided on the front surface of the housing 100a, but may be one provided on other outer surfaces (a side surface, an upper surface, and the like) of the housing 100a. When the user operates the start key 150a with the door constituting the container pressing part 102 closed, the particle manipulation of the magnetic particles 13 is started, and a plurality of processing steps are sequentially executed. In addition, when the user operates the skip key 150b while a plurality of processing steps are sequentially executed, part of the processing steps can be omitted and a next processing step can be executed.

5. Electrical Configuration of the Apparatus for Manipulating Magnetic Particles

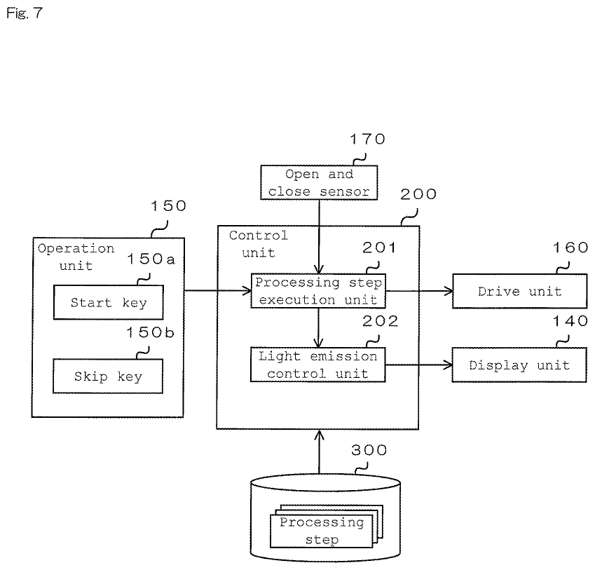

FIG. 7 is a block diagram showing an example of an electrical configuration of the apparatus 100 for manipulating magnetic particles. In addition to the display unit 140 and the operation unit 150, the apparatus 100 for manipulating magnetic particles includes a drive unit 160, an open and close sensor 170, a control unit 200, a storage unit 300, and the like.

The drive unit 160 is a mechanism for moving the magnet 130 in the vertical direction D1 along the container 20, and includes a motor or the like, for example. The open and close sensor 170 detects whether or not the door constituting the container pressing part 102 is closed. The control unit 200 has a configuration including, for example, a central processing unit (CPU), and functions as a processing step execution unit 201, a light emission control unit 202, and the like when the CPU executes a program. The storage unit 300 is configured with, for example, a random access memory (RAM), a read only memory (ROM), a hard disk, or the like, and holds details of a processing step (for example, processing content and processing time) for moving the magnet 130.

The processing step execution unit 201 controls the drive unit 160 to move the magnet 130 from the upper side to the lower side so as to execute a plurality of processing steps corresponding to positions of the magnet 130. In response to operation of the start key 150a of the operation unit 150, the processing step execution unit 201 starts control of the drive unit 160. Further, when the skip key 150b is operated while a plurality of processing steps are sequentially executed, the processing step execution unit 201 controls the drive unit 160 to move the magnet 130 so as to execute a processing step next to a processing step being executed.

In a case where the door constituting the container pressing part 102 is opened while a plurality of processing steps are sequentially executed, the processing step execution unit 201 stops control of the drive unit 160 based on a detection signal from the open and close sensor 170 and stops a processing step being executed. After the above, when the door is closed, the processing step execution unit 201 may restart the control of the drive unit 160 based on a detection signal from the open and close sensor 170.

The light emission control unit 202 controls the operation of the display unit 140 to individually cause the plurality of light emitting regions 140a to 140c to emit light. In the present embodiment, the light emitting region 140a corresponds to the dissolving step, the light emitting region 140b corresponds to the cleaning step, and the light emitting region 140c corresponds to the eluting step. That is, the light emitting regions 140a to 140c corresponding to the respective processing steps to be sequentially executed are arranged in the same order as the execution order of the respective processing steps (see FIG. 6). When a plurality of processing steps are sequentially executed, the light emission control unit 202 sequentially causes the light emitting regions 140a to 140c corresponding to the respective processing steps to emit light.

In this manner, when a plurality of processing steps corresponding to positions of the magnet 130 are sequentially executed, the light emitting regions 140a to 140c sequentially caused to emit light corresponding to the respective processing steps are visually recognizable from the outside. Therefore, even when the position of the magnet 130 is not visually recognizable from the outside, it is possible to determine a type of a processing step being executed.

Specifically, the light emitting regions 140a to 140c corresponding to a processing step being executed are lit, and when a next processing step is executed, the light emitting regions 140a to 140c corresponding to the processing step are lit, and the light emitting regions 140a to 140c corresponding to processing steps that have already been executed are also maintained in a lit state. Therefore, only the light emitting region 140a is lit during the dissolving step, the light emitting regions 140a and 140b are lit during the cleaning step, and the light emitting regions 140a to 140c are lit during the eluting step.

Alternatively, the configuration may be such that only the light emitting regions 140a to 140c corresponding to a processing step being executed are lit, and the light emitting regions 140a to 140c corresponding to a processing step that has already been executed are turned off. Further, each of the light emitting regions 140a to 140c is not limited to one that is lit, but may be configured to emit light in other modes, such as blinking.

In particular, in the present embodiment, even if a position of the magnet 130 is not visually recognizable from the outside, it can be determined whether or not a processing step (cleaning step) for removing impurities by a cleaning solution is being executed. Therefore, it is also possible to accurately determine a timing at which the cleaning processing is performed and the timing at which the cleaning process is not performed, and to take out the container 20 in the middle of any of the processing steps.

The light emission control unit 202 may cause a plurality of light emitting regions 140a to 140c to emit light in different modes. For example, when a specific processing step determined in advance as a processing step which should not be stopped halfway among a plurality of processing steps to be sequentially executed is executed, the light emitting regions 140a to 140c corresponding to the specific processing step may emit light in a mode different from that of the light emitting regions 140a to 140c corresponding to other processing steps. In this manner, it is possible to easily determine whether a processing step being executed is a processing step which should not be stopped halfway or another processing step based on a light emission mode of the light emitting regions 140a to 140c. Therefore, during execution of the processing step which should not be stopped halfway, the container 20 can be prevented from being taken out after the processing step is erroneously stopped.

The processing step which should not be stopped halfway, which is optional, may be, for example, a step of reciprocating the magnet 130 in the vertical direction D1 at a high speed, or the like. Further, the different mode is preferably a light emitting mode more user-friendly than that of the light emitting regions 140a to 140c corresponding to other processing steps, such as lighting with different colors or blinking at short intervals.

In the present embodiment, the light emitting regions 140a to 140c are arranged side by side in the lateral direction. However, the arrangement of the light emitting regions 140a to 140c is not limited to the above, and the light emitting regions 140a to 140c may be arranged side by side along other directions, such as the vertical direction. Further, in a case where the cleaning step is performed in a plurality of liquid layers 11, light emitting regions may be provided separately in association with the cleaning step in each of the liquid layers 11.

In a case where the skip key 150b is operated while a plurality of processing steps are sequentially executed and the next processing step is executed, the light emission control unit 202 causes the light emitting regions 140a to 140c corresponding to the processing step to emit light. That is, the light emitting regions 140a to 140c caused to emit light by the light emission control unit 202 and the processing step being executed always correspond to each other even when the skip key 150b is operated.

As described above, in the present embodiment, by operating the operation unit 150 (skip key 150b), part of a plurality of processing steps sequentially executed can be omitted and the next processing step can be executed. When the next processing step is executed, the light emitting regions 140a to 140c corresponding to the processing step emit light. Accordingly, even if part of processing steps are omitted, it is possible to accurately determine a type of a processing step being executed.

6. Variation

In the above embodiment, a case where part of the plurality of liquid layers 11 is a layer composed of a cleaning solution for removing impurities other than the target substance is described. However, the liquid constituting the liquid layer 11 is optional, and part of a plurality of liquid layers 11 may be, for example, a layer made of a reagent acting on the target substance. Examples of reagents include enzymes, such as a restriction enzyme for fragmenting nucleic acids and a reaction enzyme for reacting fragmented nucleic acids, but the reagents are not limited to enzymes and may be other reagents.

In this case, even if a position of the magnet 130 is not visually recognizable from the outside, whether or not a processing step (reagent step) for causing the reagent to act on the target substance is being executed can be determined. Therefore, it is also possible to accurately determine a timing at which the reagent step is being performed and a timing at which the reagent step is not performed, and to take out the container 20 in the middle of any of the processing steps.

The reagent step may include a restriction enzyme step in which the restriction enzyme acts on the target substance as a reagent and a reaction enzyme step in which the reaction enzyme acts on the target substance as a reagent. In this case, it is possible to accurately determine the timing at which the restriction enzyme step is carried out and the timing at which the reaction enzyme step is performed by causing the light emitting regions corresponding to the restriction enzyme step and reaction enzyme step to sequentially emit light. Therefore, it is also possible to take out the container 20, for example, after the restriction enzyme step is performed and before the reaction enzyme step is performed.

In the case where the reagent step includes the restriction enzyme step and the reaction enzyme step as described above, some users may desire to cause a nucleic acid to react with the reaction enzyme without fragmenting the nucleic acid by executing the reaction enzyme step while the restriction enzyme step is omitted. In such a case, by operating the operation unit 150 (skip key 150b), the reaction enzyme step can be executed while the restriction enzyme step is omitted. Even in such a case, by visually recognizing the light emission in the light emitting region, it can be accurately determined that the reaction enzyme step is being executed while the restriction enzyme step is omitted.

What is claimed is:

1. An apparatus for manipulating magnetic particles for moving magnetic particles in a state where a target substance is fixed to the magnetic particles in a tubular device in which a gel-like medium layer and a liquid layer are alternately stacked in a container and in which magnetic particles are loaded, the apparatus comprising:
a container holding part configured to hold the container;
a magnet that is disposed close to and outside the container held by the container holding part and moves the magnetic particles in the container by a magnetic force by relatively moving along the container;
a drive unit configured to relatively move the magnet along the container;
a housing configured to accommodate the magnet and the container holding part in the housing in a state in which a position of the magnet that relatively moves along the container is not visually recognizable from the outside;
a display unit that is provided on the housing so as to be visually recognizable from outside and has a light emitting region capable of emitting light;
a storage unit configured to hold details of a processing step of relatively moving the magnet;
a processing step execution unit configured to sequentially execute a plurality of processing steps by relatively moving the magnet; and
a light emission control unit configured to cause the light emitting region to emit light in accordance with a position of the magnet when the plurality of processing steps are sequentially executed.

2. The apparatus for manipulating magnetic particles according to claim 1 for a tubular device, wherein the liquid layer includes a layer made of a cleaning solution for removing impurities other than the target substance.

3. The apparatus for manipulating magnetic particles according to claim 1 for a tubular device, wherein the liquid layer includes a layer made of a reagent acting on the target substance.

4. The apparatus for manipulating magnetic particles according to claim 1, wherein the display unit has a plurality of the light emitting regions that can emit light individually.

5. The apparatus for manipulating magnetic particles according to claim 4, wherein the display unit is configured with the plurality of light emitting regions arranged side by side.

6. The apparatus for manipulating magnetic particle according to claim 4, wherein the light emission control unit causes the light emitting regions corresponding to the respective processing steps to sequentially emit light when the plurality of processing steps are sequentially executed.

7. The apparatus for manipulating magnetic particle according to claim 6, further comprising
an operation unit that is operated to execute a next processing step while part of processing steps is omitted among the plurality of processing steps sequentially executed by the processing step execution unit,
wherein when a next processing step is executed by operation of the operation unit, the light emission control unit causes a light emitting region corresponding to the processing step to emit light.

8. The apparatus for manipulating magnetic particle according to claim 6, wherein when a specific processing step determined in advance as a processing step that should not be stopped halfway among the plurality of processing steps sequentially executed by the processing step execution unit is executed, the light emission control unit causes the light emitting region corresponding to the specific processing step to emit light in a mode different from that of the light emitting regions corresponding to other processing steps.
